(19) United States
(12) Patent Application Publication
(10) Pub. No.: US 2016/0258951 A1
(43) Pub. Date: Sep. 8, 2016

(54) METHODS OF MONITORING IMMUNE RESPONSES

(71) Applicant: THE HENRY M. JACKSON FOUNDATION FOR THE ADVANCEMENT OF MILITARY MEDICINE, INC., Bethesda, MD (US)

(72) Inventors: Sathibalan Ponniah, Columbia, MD (US); George E. Peoples, San Antonio, TX (US); Catherine Storrer, Columbia, MD (US)

(21) Appl. No.: 15/032,820

(22) PCT Filed: Oct. 28, 2014
(86) PCT No.: PCT/US14/62645

§ 371 Date: Apr. 28, 2016

Related U.S. Application Data
(60) Provisional application No. 61/896,625, filed on Oct. 28, 2013.

(51) Int. Cl. G01N 33/569

(52) U.S. Cl. CPC G01N 33/56966; G01N 2800/52

(57) ABSTRACT

The human HLA-A2:1g dimer molecule is a recombinant protein comprising a mouse IgG antibody fused with two human MHC Class I HLA-A2 molecules. Any peptide (usually 8-10 amino acids in length) can be loaded into the peptide-binding groove of the two HLA molecules, for example, by incubating a mixture of the dimer and peptide solution overnight. The resulting N peptide-specific HLA-A2:1g dimer mixture can then be added to PBMCs from peripheral blood samples in order to detect CD8 T lymphocytes which express T cell receptors that are capable of specifically interacting with and binding to the peptide HLA-A2:1g dimer molecules. The presence of such specific binding activity and interactions can then be detected by additional staining with fluorescence-conjugated antibodies.

### CROSS REFERENCE TO RELATED APPLICATIONS

This application claims the benefit of, and relies on the filing date of, U.S. provisional patent application No. 61/896,625, filed 28 Oct. 2014, the entire disclosure of which is incorporated herein by reference.

### GOVERNMENT INTEREST

This invention was made in part with U.S. Government support. The U.S. Government has certain rights in the invention.

### SEQUENCE LISTING

The instant application contains a Sequence Listing which has been submitted electronically in ASCII format and is hereby incorporated by reference in its entirety. Said ASCII copy, created on Oct. 22, 2014, is named HMJ-148-PCT_SL.txt and is 12,372 bytes in size.

### BACKGROUND

Breast cancer (BCa) is the most common cancer diagnosis in women and the second-leading cause of cancer-related death among women (Ries L A G, et al. (eds.). SEER Cancer Statistics Review, 1975-2003, National Cancer institute, Bethesda, Md.). Major advances in breast cancer treatment over the last 20 years have led to significant improvement in the rate of disease-free survival (DFS). For example, therapies utilizing antibodies reactive against tumor-related antigens have been used to block specific cellular processes in order to slow disease progress or prevent disease recurrence. Despite the recent advances in breast cancer treatment, a significant number of patients will ultimately die from recurrent disease.

Vaccines are an attractive model for preventing, slowing, or prohibiting the development of recurrent disease due to their ease of administration, and because of their high rate of success observed for infectious diseases. The basic concept of constructing a cancer vaccine is straightforward in theory. The development of effective cancer vaccines for solid tumors in practice, however, has met with limited success. For example, one group attempting to administer a peptide vaccine directed against metastatic melanoma observed an objective response rate of only 2.6% (Rosenberg S A et al. (2004) Nat. Med. 10:909-15).

There are many potential explanations for this low success rate (Campoli M et al. (2005) Cancer Treat. Res. 123:61-88). For example, even if an antigen is specifically associated with a particular type of tumor cell, the tumor cells may express only low levels of the antigen, or it may be located in a cryptic site or otherwise shielded from immune detection. In addition, tumors often change, their antigenic profile by shedding antigens as they develop. Also contributing to the low success rate is the fact that tumor cells may express very low levels of MHC proteins and other co-stimulatory proteins necessary to generate an immune response.

Additional problems facing attempts at vaccination against tumors arise in patients with advanced-stage cancers. Such patients tend to have larger primary and metastatic tumors, and the cells on the interior of the tumor may not be accessible due to poor blood flow. This is consistent with the observation that vaccine strategies have tended to be more successful for the treatment of hematologic malignancies (Radford K J et al. (2005) Pathology 37:534-50; and Molldrem J J (2006) Biol. Bone Marrow Transplant. 12:13-8). In addition, as tumors become metastatic, they may develop the ability to release immunosuppressive factors into their microenvironment (Campoli, 2005; and, Kortylewski M et al. (2005) Nature Med. 11:1314-21). Metastatic tumors have also been associated with a decrease in the number of peripheral blood lymphocytes, and dendritic cell dysfunction (Gillanders W E et al. (2006) Breast Diseases: A Year Book and Quarterly 17:26-8).

While some or all of these factors may contribute to the difficulty in developing an effective preventative or therapeutic vaccine, the major underlying challenge is that most tumor antigens are self antigens or have a high degree of homology with self antigens, and are this expected to be subject to stringent immune tolerance. Thus, it is clear that many peptide-based cancer vaccines, with or without immune-stimulating adjuncts, may be doomed to only limited success in clinical practice due to low immunogenicity and lack of specificity.

Prototype breast cancer vaccines based on single antigens have been moderately successful in inducing a measurable immune response in animal experiments and in clinical tests with breast cancer patients. The observed immune response, however, has not translated into a clinically-significant protective immunity against recurrence of disease put in remission by standard therapy (e.g., surgery, radiation therapy, and chemotherapy).

HER2/neu is a proto-oncogene expressed in many epithelial malignancies (Slamon D J et al. (1989) Science 244:707-12). HER2/neu is a member of the epidermal growth factor receptor family and encodes a 185-kd tyrosine kinase receptor involved in regulating cell growth and proliferation. (Popescu N C et al. (1989) Genomics 4:362-366; Yarden Y at al. (2001) Nat Rev Mol Cell Bio 2:127-13T) Over-expression and/or amplification of HER2/neu is found in 25-30% of invasive breast cancers (BCa) and is associated with more aggressive tumors and a poorer clinical outcome. (Slamon D J at al. Science (1987) 235:177-182; Slamon D J at al. Science (1989) 244:707-12; Toikkanen S et al. J Clin Oncol (1992) 10:1044-1048; Pritchard K I at al. (2006) N. Engl. J. Med. 354:2103-11.) HER2/neu overexpression and/or amplification have also been observed in ovarian cancer (Disis et al., (1999) Clin Cancer Res. 5:1289-97), prostate cancer (Yan Shi et al., (2001) J. Urology 166:1514-19), colon cancer (Saudi et al., (2006) BMC Cancer 8(6):123), bladder cancer (Eltze et al. (2005) Int J. Oncol. 26(6):1525-31), gastric cancer (Gravalos at al. (2008) Annals of Oncology 19(9):1523-29), pancreatic cancer (Safran et al. (2001) Am. J. Clin. Oncol. 24(5):496-99), non-small cell lung cancer (Yoshino (1994) Cancer Res. 54:3387-90), endometrial cancer (Hetzel at al. (1992) Gynecol. Oncol. 47:179-85), uterine cervix cancer (Mitra at al. (1994) Cancer Res. 54:637-39), esophageal cancer (Reichelt et al. (2007) Med. Path. 20(1):120-129), and head and neck squamous cell carcinoma (Beckhardt at al. (1995) 121(11):1265-70).

Determining HER2/neu status is performed predominately via two tests, immunohistochemistry (IHC) and fluorescence in situ hybridization (FISH). IHC detects over-expression of HER2/neu protein and is reported on a semi-quantitative scale of 0 to 3+ (0=negative, 1⁺=low expression, 2⁺=intermediate, and 3⁺=over-expression). FISH on the other hand detects amplification (excess copies) of the HER2/neu gene and is expressed as a ratio of HER2/neu gene copies to chromosome 17 gene copies and interpreted as “over-expression” if FISH is≧2.0 copies. (Hicks D G et al. Hum Pathol (2005) 36:250-261.) Concurrence rate of IHC and FISH is approximately 90%. (Jacobs et al. J Clin Oncol (1999) 17:1533-1541.) FISH is considered the gold standard, as retrospective analysis reveals it is a better predictor of trastuzumab (Tz) response; it is more objective and reproducible. (Press M E et at J Clin Oncol (2002) 14:3095-3105; Bartlett J et al. J Pathol (2003) 199:411-417; Wolff A C et al. J Clin Oncol (2007) 25:118-145.)

Identification and quantification of HER2/neu as a proto-oncogene has led to humoral or antibody-based passive immunotherapy, including the use of trastuzumab (Herceptin® Genentech Inc., South San Francisco, Calif.). Trastuzumab is a recombinant, humanized monoclonal antibody that binds the extracellular juxtamembrane domain of HER2/neu protein. (Plosker G L et al. Drugs (2006) 66:449475.) Tz is indicated for HER2/neu over-expressing (IHC 3⁺ or FISH≦2.0) node-positive (NP) and metastatic BCa patients, (Vogel C L et al. J Clin Oncol (2002) 20:719-726; Piccart-Gebhart M J et al. N Engl. J Med (2005) 353:1659-1672) and shows very limited activity in patients with low to intermediate HER2/neu expression. (Herceptin® (Trastuzumab) prescription product insert, Genentech Inc, South San Francisco, Calif.: revised September 2000.)

Another form of immunotherapy being pursued is vaccination and active immunotherapy targeting a cellular immune response to epitopes on tumor associated antigens, such as HER2/neu. HER2/neu is a source of several immunogenic peptides that can stimulate the immune system to recognize mid kill HER2/neu-expressing cancer cells. (Fisk B et al. J Exp Med (1995) 181:2109-2117.) Two such peptides are termed E75 and GP2. E75 and GP2 are both nine amino-acid peptides that are human leukocyte antigen (HLA)-A2-restricted and stimulate CTL to recognize and lyse HER2/neu-expressing cancer cells (Fisk B at al. J Exp Med (1995) 181:2109-2117; Peoples G E et al. Proc Natl Acad Sci USA (1995) 92:432-436). Cancer vaccines targeting “self” tumor antigens, like HER2/neu, present unique challenges because of the immunologic tolerance characteristic of self proteins.

E75 is derived from the extracellular domain of the HER2/neu protein and corresponds to amino acids 369-377 (KIFGSLAFL) (SEQ ID NO:2) of the HER2/neu amino acid sequence and is disclosed as SEQ ID NO:11 in U.S. Pat. No. 6,514,942, which patent is hereby incorporated by reference in its entirety. The full length HER2/neu protein sequence is set forth below and is disclosed as SEQ ID NO:2 in U.S. Pat. No 5,869,445, which patent is hereby incorporated by reference in its entirety:

(SEQ ID NO: 1)
MELAALCRWGLLLALLPPGAASTQVCTGTDMKLRLPASPETHLDML
RHLYQGCQVVQGNLELTYLPTNASLSFLQDIQEVQGYVLIAHNQVR
QVPLQRLRIVRGTQLFEDNYALAVLDNGDPLNNTTPVTGASPGGLR
ELQLRSLTEILKGGVLIQRNPQLCYQDTILWKDIFHKNNQLALTLI
DTNRSRACHPCSPMCKGSRCWGESSEDCQSLTRTVCAGGCARCKGP
LPTDCCHEQCAAGCTGPKHSDCLACLHFNHSGICELHCPALVTYNT
DTFESMPNPEGRYTFGASCVTACPYNYLSTDVGSCTLVCPLHNQEV
TAEDGTQRCEKCSKPCARVCYGLGMEHLREVRAVTSANIQEFAGCK
KIFGSLAFLPESFDGDPASNTAPLQPEQLQVFETLEEITGYLYISA
WPDSLPDLSVFQNLQVIRGRILHNGAYSLTLQGLGISWLGLRSLRE
LGSGLALIHHNTHLCFVHTVPWDQLFRNPHQALLHTANRPEDECVG
EGLACHQLCARGHCWGPGPTQCVNCSQFLRGQECVEECRVLQGLPR
EYVNARHCLPCHPECQPQNGSVTCFGPEADQCVACAHYKDPPFCVA
RCPSGVKPDLSYMPIWKFPDEEGACQPCPINCTHSCVDLDDKGCPA
EQRASPLTSIISAVVGILLVVVLGVVFGILIKRRQQKIRKYTMRRL
LQETELVEPLTPSGAMPNQAQMRILKETELRKVKVLGSGAFGTVYK
GIWIPDGENVKIPVAIKVLRENTSPKANKEILDEAYVMAGVGSPYV
SRLLGICLTSTVQLVTQLMPYGCLLDHVRENRGRLGSQDLLNWCMQ
IAKGMSYLEDVRLVHRDLAARNVLVKSPNHVKITDFGLARLLDIDE
TEYHADGGKVPIKWMALESILRRRFTHQSDVWSYGVTVWELMTFGA
KPYDGIPAREIPDLLEKGERLPQPPICTIDVYMIMVKCWMIDSECR
PRFRELVSEFSRMARDPQRFVVIQNEDLGPASPLDSTFYRSLLEDD
DMGDLVDAEEYLVPQQGFFCPDPAPGAGGMVHHRHRSSSTRSGGGD
LTLGLEPSEEEAPRSPLAPSEGAGSDVFDGDLGMGAAKGLQSLPTH
DPSPLQRYSEDPTVPLPSETDGYVAPLTCSPQPEYVNQPDVRPQPP
SPREGPLPAARPAGATLERPKTLSPGKNGVVKDVFAFGGAVENPEY
LTPQGGAAPQHPPPAFSPAFDNLYYWDQDPPERGAPPSTFKGTPTA
ENPEYLGLDVPV

GP2, initially described by Peoples et al., is a nine amino acid peptide derived from the transmembrane portion of the HER2/neu protein corresponding to amino acids 654-662 of the full length sequence (i.e., IISAVVGIL: SEQ ID NO:3) (Peoples G E et al., Proc Natl Acad Sci USA (1995) 92:432-436, which is hereby incorporated by reference in its entirety). The peptide was isolated using tumor-associated lymphocytes from patients with breast and ovarian cancer, and later found to be shared amongst several epithelial malignancies including non-small cell lung cancer and pancreatic cancer (Linehan D C et al., J Immunol (1995) 155:4486-4491: Peiper M et al., Surgery (1997) 122:235-242; Yoshino I et al., Cancer Res (1994) 54:3387-3390; Peiper M et al., Eur J Immunol (1997) 27:1115-1123).

E75 and GP2 are being used as clinical vaccines in patients with HER2/neu⁺ breast cancer (Peoples et al., J Clin Oncol (2005) 23:7536-7545; Mittendorf E et al., Cancer (2006) 106:2309-2317). Thus far, they have been shown to be safe and effective in stimulating antigen-specific immunity, and the immunity conferred by E75 appears to have clinical benefit in decreasing breast cancer recurrence (Peoples G E et al., Clin Cancer Res (in press)). Booster vaccinations help to sustain vaccine-induced immunity (Peoples G E et al., Clin Cancer Res (in press); Knutson K L et al., Clin Cancer Res (2002) 81014-1018). WO 2007/030771 and WO 2009/112792, also disclose compositions comprising E75 or GP2 and an antibody, such as Trastuzumab, and methods of using those compositions to treat cancer patients.

### SUMMARY

The human HLA-A2:Ig dimer molecule is a recombinant protein comprising a mouse IgG antibody fused with two human MHC Class I HLA-A2 molecules. Any peptide (usually 8-10 amino acids in length) can be loaded into the peptide-binding groove of the two FHA molecules, for example, by incubating a mixture of the dimer and peptide solution overnight at 37° C. The resulting peptide-specific HLA-A2:Ig dimer mixture can then be added to PBMCs from peripheral blood samples in order to detect CD8 T lymphocytes which express T cell receptors that are capable of specifically interacting with and binding to the peptide HLA-A2:Ig dimer molecules. The presence of such specific binding activity and interactions can then be detected by additional staining with fluorescence-conjugated antibodies and analyzing the sample using, for example, flow cytometry. This then allows one to quantitate the number of peptide-specific CD8 T lymphocytes in the blood sample. This is very useful because it allows one to monitor the number of peptide-specific CD8 T lymphocytes in samples of peripheral blood obtained at various times from one or more individuals. For example in a clinical trial investigating a HLA-A2 peptide vaccine, this assay can be used to measure the ability of the vaccination(s) to modulate (no change, increase or decrease) the number of peptide vaccine-specific CD8 T lymphocytes in the subject. The ability to obtain such information is important for optimizing the vaccine dose, schedule and formulation(s) to achieve full efficacy potential of the vaccine treatment.

The present disclosure provides methods of monitoring art immune response and kits for use in such methods.

In one embodiment, the method of monitoring an immune response to a test peptide comprises:

(a) incubating peripheral blood mononuclear cells (PBMCs) obtained from a patient with a fusion protein comprising a mouse immunoglobulin fused to two human MHC class I HLA molecules, wherein the test peptide has been loaded into the peptide binding groove of the two human MHC class I HLA molecules;

(b) quantitating the cytotoxic CD8 T cells specific for the test peptide; and

(c) normalizing the result obtained in step (b) by subtracting a background value obtained by quantitating the cytotoxic CD8 T cells specific for a negative control peptide, wherein the negative control peptide produces a stable, non-zero background immune response. In one embodiment, the negative control peptide is E37 from the folate binding protein (RIAWARTEL) (SEQ ID NO:4) and the background value is about 0.3%.

In another embodiment, the method of monitoring an immune response to a test peptide comprises:

(a) incubating peripheral blood mononuclear cells (PBMCs) obtained from a patient with a fusion protein comprising a mouse immunoglobulin fused to two human MHC class I HLA molecules, wherein the test peptide has been loaded into the peptide binding groove of the two human MHC class I HLA molecules;

(b) incubating PBMCs obtained from the patient with the fusion protein, wherein a negative control peptide has been loaded into the peptide binding groove of the two human MHC class I HLA molecules, and wherein the negative control peptide produces a stable, non-zero background immune response;

(c) quantitating the cytotoxic CD8 T cells specific for the test peptide;

(d) quantitating the cytotoxic CD8 T cells specific for the negative control peptide to obtain a background value;

(e) normalizing the result obtained in step (c) by subtracting the background value obtained in step (d). In one embodiment, the negative control peptide is E37 from the folate binding protein (RIAWARTEL) (SEQ ID NO:4).

In one embodiment, the test peptide is a class I restricted Her2/neu derived peptide, including, but not limited to E75 (KIFGSLAFL) (SEQ ID NO:2), GP2 (IISAVVGIL) (SEQ ID NO:3) GP2′ (IVSAVVGIL) (SEQ ID NO:5), or Her⁵⁷⁷ (FGPEADQCV) (SEQ ID NO:6). In one embodiment the fusion protein comprises a mouse IgG fused to two human MHC class I HLA-A2 molecules. In another embodiment, quantitating the cytotoxic T cells specific for the test peptide is achieved using immunofluorescent staining or fluorescent activated cell sorting (FACS). In yet another embodiment, quantitating the cytotoxic CD 8 T cells specific for the test peptide is expressed as a dimer index.

In one embodiment, the method further comprises a step before (a) of loading the test peptide into the peptide binding groove of the two human MHC class I MLA molecules of the fusion protein. In one embodiment, the loading step comprises incubating the test peptide with the fusion protein overnight at 37° C.

In one aspect, the method is used to assess the clinical response of a HER2/neu derived peptide vaccine in a breast cancer patient, wherein the breast cancer patient is disease free following standard therapy. In one embodiment, the HER2/neu derived peptide vaccine comprises E75 and GM-CSF. In another embodiment, the HER2/neu derived peptide vaccine comprises GP2 and GM-CSF. In another embodiment, the HER2/neu derived peptide vaccine comprises GP2′ and GM-CSF. In another embodiment, the HER2/neu derived peptide vaccine comprises Her⁵⁷⁷ and GM-CSF.

In one embodiment, the cytotoxic CD8 T cells specific for the test peptide are quantified at baseline, after a primary vaccination, and at 6 months post primary vaccination. In yet another embodiment, a more robust cytotoxic CD8 T cell response to the test peptide at 6 months post primary vaccination indicates that the patient is less likely to experience breast cancer recurrence or more likely to have a longer disease free survival. In another embodiment, a low cytotoxic CD8 T cell response to the test peptide at baseline indicates that the patient is less likely to experience breast cancer recurrence or more likely to have a longer disease free survival. As described in the application, the HLA-A2:Ig dimer assay can be used to quantitate the cytotoxic CD8 I cell response using a mean dimer index (mdi). In certain embodiments, a low cytotoxic CD8 T cell response is a lower than average mdi. In other embodiments, a more robust cytotoxic CD8I cell response refers to a higher than average mdi. In one embodiment, the patient has low to intermediate HER2 expression (IHC 1 or 2+ or FISH<2.2).

Another aspect is directed to a kit for use in a method of monitoring an immune response. In one embodiment, the kit comprises a fusion protein comprising a mouse immunoglobulin fused to two human MHC class I HLA molecules and a negative control peptide, wherein the negative control peptide produces a stable, non-zero background immune response. In one embodiment, the negative control peptide is E37 from the folate binding protein and consists of the amino acid sequence RIAWARTEL (SEQ ID NO:4). In another embodiment, the E37 peptide is loaded into the peptide binding groove of the two human MHC class I HLA molecules of the fusion protein. In one embodiment, the fusion protein comprises a mouse IgG fused to two human MHC class I HLA-A2 molecules. In another embodiment, the kit comprises a positive control peptide, including for example, the Flu M peptide.

The kit optionally contains a test peptide. In one embodiment, the test peptide is a class I restricted Her2/neu derived peptide, including, but not limited to E75, GP2, GP2′, or Her⁵⁷⁷. In one embodiment, the kit comprises a first container comprising the fusion protein, wherein the test peptide is loaded into the peptide binding groove of the two human MHC class I HLA molecules of the fusion protein and a second container comprising the fusion protein, wherein the E37 peptide is loaded into the peptide binding groove of the two human MHC class I HLA molecules of the fusion protein.

In another embodiment, the kit further comprises a labeled antibody that binds to the immunoglobulin of the fusion protein. In one embodiment, the labeled antibody binds to mouse immunoglobulin, including, for example, mouse IgG. In another embodiment, the kit further comprises a buffer for diluting one or more of the negative control peptide, positive control peptide or test peptide.

### BRIEF DESCRIPTION OF THE DRAWINGS

The accompanying drawings, which are included to provide a further understanding of the invention and are incorporated in and constitute a part of this specification, illustrate aspects of the invention and together with the description serve to explain the principles of the invention. In the drawings:

FIG. 1 shows the cytotoxic T cell clonal expansion as measured by the dimer assay in breast cancer patients after receiving the primary vaccine series (R6) of E75+GM-CSF (Neu Vax).

FIG. 2 shows the disease free-survival (DFS) of a subset of breast cancer patients after receiving the primary vaccine series (R6) of E75+GM-CSF (Neu Vax), the subset having R6 dimer measurements above the mean.

FIG. 3 shows the maximum dimer change (between baseline and maximum mdi) in a subset of breast cancer patients who under express HER2.

FIG. 4 shows the disease free-survival (DFS) of a subset of breast cancer patients after receiving the primary vaccine series (R6) of E75+GM-CSF (Neu Vax), the subset having a delta max dimer above the mean.

### DETAILED DESCRIPTION

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The term “prevent” or “prevention” refers to any success or indicia of success in the forestalling or delay of cancer recurrence/relapse in patients in clinical remission, as measured by any objective or subjective parameter, including the results of a radiological or physical examination.

“Effective amount” or “therapeutically effective amount” are used interchangeably herein, and refer to an amount of a compound, material, or composition, as described herein effective to achieve a particular biological result such as, but not limited to, biological results disclosed, described, or exemplified herein. Such results may include, but are not limited to, the prevention of cancer, and more particularly, the prevention of recurrent cancer, e.g., the prevention of relapse in a subject, as determined by any means suitable in the art. Optimal therapeutic amount refers to the dose, schedule and the use of boosters to achieve the best therapeutic outcome.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

“Protective immunity” or “protective immune response,” means that the subject mounts an active immune response to an immunogenic component of an antigen such as the breast cancer antigens described and exemplified herein, such that upon subsequent exposure to the antigen, the subject's immune system is able to target and destroy cells expressing the antigen, thereby decreasing the incidence of morbidity and mortality from recurrence of cancer in the subject. Protective immunity in the context of the present invention is preferably, but not exclusively, conferred by T lymphocytes.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Peptide” refers to any peptide comprising two or more amino acids joined to each other by peptide bonds or modified peptide bonds, i.e., peptide isosteres. Polypeptide refers to both short chains, commonly referred to as peptides, oligopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from natural posttranslational processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristaylation, oxidation, proteolytic processing, phosphorylation, prenylation, racemization, selenoylation, sulfation, transfer-RNA mediated addition of amino acids to proteins such as arginylation, and ubiquitination.

“Booster” refers to a dose of an immunogen administered to a patient to enhance, prolong, or maintain protective immunity and to overcome the down-regulation of T-cell responses mediated by regulatory T-cells.

“Free of cancer” or “disease free” or NED (No Evidence of Disease) means that the patient is in clinical remission induced by treatment with the current standard of care therapies. By “remission” or “clinical remission,” which are used synonymously, it is meant that the clinical signs, radiological signs, and symptoms of cancer have been significantly diminished or have disappeared entirely based on clinical diagnostics, although cancerous cells may still exist in the body. Thus, it is contemplated that remission encompasses partial and complete remission. The presence of residual cancer cells can be enumerated by assays such as CTC (Circulating Tumor Cells) and may be predictive of recurrence.

“Relapse” or “recurrence” or “resurgence” are used interchangeably herein, and refer to the radiographic diagnosis of return, or signs and symptoms of return of cancer after a period of improvement or remission.

The chimeric MHC:immunoglobulin reagents described in this application can be used to monitor an immune response, for example, by quantitating the number of peptide-specific CD8 T cells in a peripheral blood sample obtained from a patient, such as a patient undergoing a vaccine or other therapeutic regimen. Obtaining such information can be important for optimizing a dose, schedule and/or formulation(s) to achieve full efficacy potential of the treatment, including in cancer immunotherapy.

However in order for this reagent and assay to be of any real clinical or scientific value a number of additional aspects have to be discovered. First, dimers loaded with a negative control peptide should be used in order to determine the level of background or non-specific binding of dimer molecules to CD8 T lymphocytes (due to non-specific interactions between the recombinant dimer protein and all other membrane proteins expressed on the surface of the CD8 T cells) in each sample. A good or standard negative control peptide would be one that has similar/significant binding affinity equal/comparable to that of the test peptide and will consistently/reliably provide a low background level of staining in almost any sample being tested. The identification of such a peptide for this purpose can often be a rare/arduous event since the immune system is geared towards the recognition of many peptides. None of the other similar technologies have identified a reliable negative standard peptide (that can perform consistently) as we have that has been used for as large a number of samples as we have done. Having identified such a peptide, we were able to establish a standard value or range of values that can be assigned as the negative or background staining value for almost any sample being tested using the negative peptide-loaded dimers. This is yet another arduous task again simply because a certain amount of non-specific interactions or background binding between proteins is always going to be present in exposing immune cells to recombinant proteins.

One such negative control peptides is the E37 peptide from the folate binding protein, NH2-RIAWARTEL-COOH (SEQ ID NO 4). Using E37, we have been able to assign a standard value for background binding due to or associated with the use of this peptide in the staining of almost 2000 peripheral blood samples from about 200-300 patients. The unique approach is to set the negative control E37 values at 03% (i.e. 0.25-0.34 range) and everything else i.e. value associated with any other peptide-specific dimer is read off relative to this “gold standard.” The assignment of a fixed value for a negative standard has never been proposed in a method for using chimeric MHC:immunoglobulin molecules in monitoring immune responses.

Secondly, the loading of peptides is preferably done by a ‘passive’ incubation process at 37 C, a process which is known to occur physiologically in the case of antigen presenting cells taking up (being pulsed) with antigenic peptides. In the case of the other similar technologies the loading of peptides is done in an artificial method whereby the recombinant HLA molecules are denatured and unfolded by altering the pH of the solution and then refolding the polypeptide/protein in the presence of the peptide(s) of interest and assuming that the resulting HLA molecule structure still resembles naturally occurring HLA molecules. However it is known that this is not how peptides are loaded on to HLA molecules in normal human antigen presentation pathways.

Using this approach, we have found that the E75-or GP2-peptide loaded dimers are able to provide realistic/significant results in detecting increased amounts of vaccine-specific CD8 T cells only in patients receiving the peptide vaccine (E75+GMCSF or GP2+GMCSF) and not in those patients receiving only GMCSF in the placebo arm of our clinical trials as would be expected/predicted. These observations provide validation of our assay and unique/novel analytical approach. In all of these samples the results obtained for using the positive control dimers (containing a peptide—Flu M—from the matrix protein of the influenza protein) have always turned out to be significantly higher or positive when compared to the background value.

Further analysis of the levels of E75-specific CD8 T lymphocytes in the breast cancer patients in our clinical trials have indicated that using our unique approach of analyzing flow cytometry data from the dimer assay has potential clinical value in its use as a risk factor assessment of patients who would experience recurrence of disease as well as those who will benefit from receiving a HER2 peptide derived vaccine as an immunotherapy.

Finally, dimers loaded with a positive control peptide should be used in every assay i.e. a HLA-A2 specific peptide with significant binding affinity and universally accepted as being recognizable by a significant number of CD8 T lymphocytes in all HLA-A2

1. Negative Control Peptide

It is useful to include a negative control peptide in the assay and, in particular, short peptides that produce a stable, non-zero-background response in an immunological assay, such as those described in U.S. Pat. No. 8,133,691, which is hereby incorporated by reference in its entirety. These short peptides may be of any size that allows for association with MHC molecules for presentation to a cell population to elicit an immune response. Peptides of the invention preferably contain a single peptide sequence from 6 to 36 amino acids in length, and preferably contain at least two anchor amino acids. Generally, the larger the sequence, the greater the number of anchor amino acids necessary for association with the appropriate MHC molecule for presentation. More preferably, the peptides are from 8 to 25 amino acids in length, with from 2 to 5 anchor amino acids. Even more preferably, sequences are from 10 to 20 amino acids, and contain from 2 to 4 anchor amino acids. Peptide sequences of 7, 9, 11, 12, 13, 14, 15, 16, 17, 18, 19, 21, 22, 23, 24, 26, 27, 28, 29, 30, 31, 32, 33, 34 and 35 amino acids are also contemplated, containing any of 1, 2, 3, 4, 5, 6, or more anchor amino acids. Although sequences will mostly contain naturally occurring amino acids, one or more (or all) non-naturally occurring, synthetic and/or modified amino acids may also comprise the peptide sequence of the invention. Amino acids may be modified or coupled with other molecules, provided the peptide is able to elicit some measure of an immune response. In a preferred embodiment of the invention, the short peptide has a sequence of 8 to 10 naturally occurring amino acids and contains two anchor amino acids, at least one at position 1, 2 or 3, and at least one other at position 7, 8, 9 or 10 (when counting from the N-terminus to the C-terminus of the peptide). Another preferred embodiment is a peptide of from 7 to 24 amino acids, with at least one anchor amino acid at any of positions 2-5 from the N-terminus, and at least one other anchor ammo acids at any of positions 2-7 from the C-terminus. Other preferred locations of anchor amino acids along the peptide chain are between amino acid positions 12 and 17 (counting from the N-Terminus), with peptides of from 22 to 28 amino acids, and between amino acid positions 22 and 25, with peptides of from 30 to 36 amino acids.

Anchor amino acids can be identified for most any peptide by those skilled in the art. For example, U.S. Patent Application Publication No. 2004 0157273, which was published Aug. 12, 2004 (and is entirely incorporated by reference), provides methods whereby amino acids of a peptide sequence with a high affinity to MHC antigen can be identified. Coefficients of affinity can be determined for such peptides for use in the development of algorithms for the prediction of specific binding sites of a peptide.

Investigations of the crystal structure of the human MHC class I molecule, HLA-A2.1, show that a peptide binding groove is created by the folding of the alpha. 1 and alpha.2 domains of the class I heavy chain (Bjorkman, et al., Nature 329:506 (1987)). Buus, et al., Science 242:1065 (1988), described a method for acid elution of bound peptides from MHC. Subsequently, Rammensee and his coworkers (Falk, et al., Nature 351:290 (1991)), developed an approach to characterize naturally processed peptides bound to class I molecules. Other investigators have successfully achieved direct amino acid sequencing of the more abundant peptides in various HPLC fractions by conventional automated sequencing of peptides eluted from class I molecules of the B type (Jardetzky, et al., Nature 353:326 (1991)) and of the A2.1 type by mass spectrometry (Hunt, et al., Science 225:1261 (1992)). A review of the characterization of naturally processed peptides in MHC Class I has been presented by Rotzschke & Falk (Rotzschke & Falk, Immunol. Today 12:447 (1991)). PCT publication WO 97/34621, incorporated herein by reference, describes peptides which have a binding affinity for A2.1 alleles. Sette, et al., Proc. Nat'l. Acad. Sci. USA 86:3296 (1989) showed that MHC allele specific motifs can predict MHC binding capacity. Schaeffer, et al., Proc. Nat'l. Acad. Sci, USA 86:4649 (1989), showed that MHC binding was related to immunogenicity. Others (De Bruijn, et al., Eur. J. Immunol., 21:2963-2970 (1991); Pamer, et al., 991 Nature 353:852-955 (1991)), provided preliminary evidence that class I binding amino acids can be applied to the identification of potential immunogenic peptides in animal models. The combined frequencies of these different alleles should be high enough to cover a large fraction or perhaps the majority of the human outbreed population. From these and other investigations, all well-known by those skilled in the art, the identity of amino acids bound to the groove, which in most cases is the high affinity binding site, of an MHC molecule can be determined. Most preferably, these are the anchor amino acids.

In one embodiment, the negative control peptide is E37 from the folate-binding protein (FBP): NH2-RIAWARTEL-COOH (SEQ ID NO 4).

2. Multivalent, Chimeric MHC:Immunoglobulin Proteins

The methods disclosed herein use a chimeric protein that is able to stably hind and modulate antigen-specific CD8 T cells, such as the chimeric proteins disclosed in U.S. Pat. No 6,268,411, which is hereby incorporated by reference in its entirety. Likewise, the chimeric MHC:immunoglobulin molecules disclosed in Greten et al., PNAS, 95:7568-73 (1998); Sakai et al., Blood, 98(5):1506-11 (2001); Nagai et al., J. Infect. Dis. 183:197-205 (2001); Greten and Scheck. Clinical and Diagnostic Laboratory Immunology, 9(2):216-20 (2002); and Fahmy et al., J. Immunol. Methods, 268:93-106 (2002), all of which are hereby incorporated by reference in their entirety, can be used in the disclosed methods for monitoring an immune response.

In one embodiment, the chimeric protein is a recombinant human Class I MHC:immunoglobulin dimer molecule comprising a mouse antibody fused with two human MHC Class I molecules—one at each antigen binding site of the antibody. In one embodiment, the mouse antibody is an IgG antibody. In another embodiment, the two human MHC Class I molecules are HLA-A2 molecules.

Any peptide (usually 8-10 amino acids in length) with sufficient binding affinity to HLA-A2 (determined by T2 cell binding assay or computer prediction algorithms) can be loaded into the peptide-binding groove of the two HLA molecules. In one embodiment, the peptide is loaded by incubating a mixture of the dimer and peptide solution overnight at 37° C. In one embodiment, the peptide is a Her2/neu derived peptide, including but not limited to, E75, GP2, GP2′, or Her⁵⁷⁷.

The following examples are provided to describe the invention in greater detail. They are intended to illustrate, not to limit, the invention.

### EXAMPLE 1

### Use of HLA-A2:Ig Dimer Assay to Predict Clinical Benefit of E75 GM-CSF Vaccine

Whether adjuvant cancer vaccines work by inducing an immune response (IR) vs augmenting a pre-existing IR is unknown. We have completed 5-yr follow-up of a phase II trial with NeuVax (nelipepimut-S or E75); an HLA-A2/A3-restricted, HER2-derived vaccine, administered in the adjuvant setting to prevent recurrence in breast cancer patients rendered disease-free after standard therapy. Using logistical regression modeling (LRM), we determined the best IR parameter for predicting disease-free survival (DFS) after completion of the primary vaccine series (PVS) to address the debate over pre-existing IR.

HLA-A2/A3+ breast cancer patients with any level of HER2 (IHC 1-3+) were enrolled into the vaccine group (VG). HLA-A2/A3-patients were followed prospectively as a control group (CG). The VG received 4-6 monthly inoculations of NeuVax+GM-CSF during the PVS. A voluntary booster program offered up to 4 inoculations every 6-months post-PVS. In-vitro IR was assessed for E75-specific, CD8+ T cell clonal expansion by the dirtier assay pre-vaccination (R0), after PVS (R6), and 6-months after the PVS (RC6). In-vivo IR was assessed by delayed type hypersensitivity (DTH) reactions to E75 at baseline (DTH1) and post-PVS (DTH2). A LRM with backwards elimination of in-vitro/in-vivo tests was used to predict recurrence. Odds ratio and the area-under-the-curve (AUC) from ROC curves was reported for statistical analysis.

Of 195 patients enrolled, 8 withdrew, leaving 108 VG and 79 CG patients.

| | Variables | | | Area |
|---|---|---|---|---|
| | in the | R0 Odds | | Under |
| Sample | Optimal | Ratio (p- | Sensitivity, | the |
| (n=) | Model | value) | Specificity | Curve |
| Vaccine Group (n = | R0, RC6 | 2.36 (0.09) | 75%, 73% | 0.71 |
| 93) | | | | |
| HER 2 Low-to | R0 | 1.90 (0.19) | 80%, 83%* | 0.72 |
| Intermediate | | | (R0 < 1.19) | |
| Expression (IHC 1 | | | | |
| or 2+, FISH <2.2), | | | | |
| (n = 58) | | | | |
| Unboosted (n = 52) | R0, DTH | 3.37 (0.12) | 85%, 70% | 0.76 |

Patients lacking pre-existing immunity, exhibiting a low R0-dimer (R0<1.10 in HER2 Low-to Intermediate Expression), have improved DFS after Neu Vax. Thus, lower pre-existing peptide-specific CTL levels correlate with fewer recurrences of cancer. This suggests that induction, rather than amplification, of an anti-HER2 IR is most beneficial clinically and may partially explain why NeuVax works best in HER2 1+/2+ patients with less HER2 antigen exposure.

Ex vivo immune response was assessed for E75-specific, cytotoxic T lymphocyte clonal expansion by HLA-A2:IgG dimer assay and expressed as mean dimer index (mdi) at baseline, after the primary vaccine series (R6), and six months after the primary vaccine series. HER2 under-expression was defined as an IHC 1+ or 2+, and a FISH<2.2. The vaccine group and control group were followed for clinical recurrence over 60 months. P-values were calculated using the Fisher's exact or by Log-Rank test.

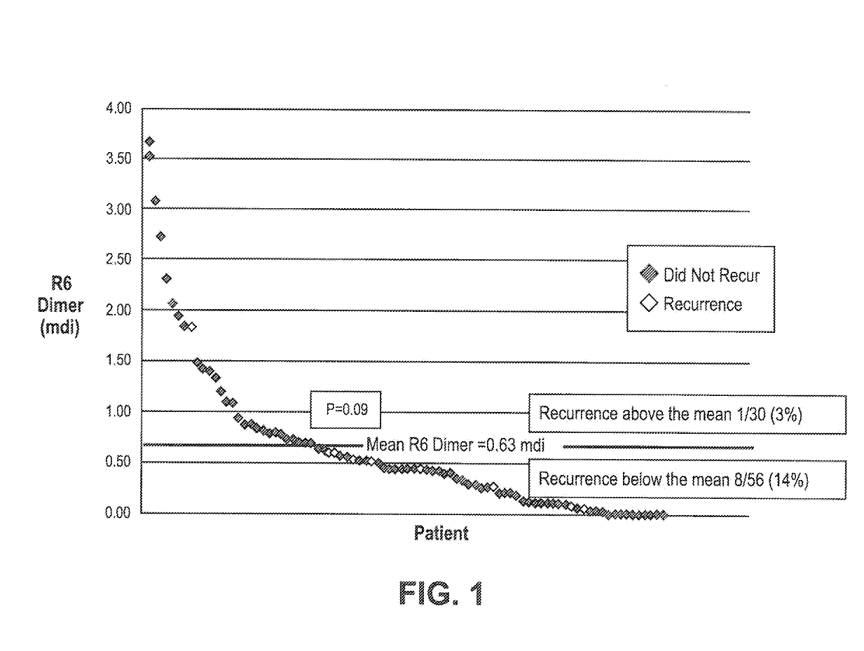

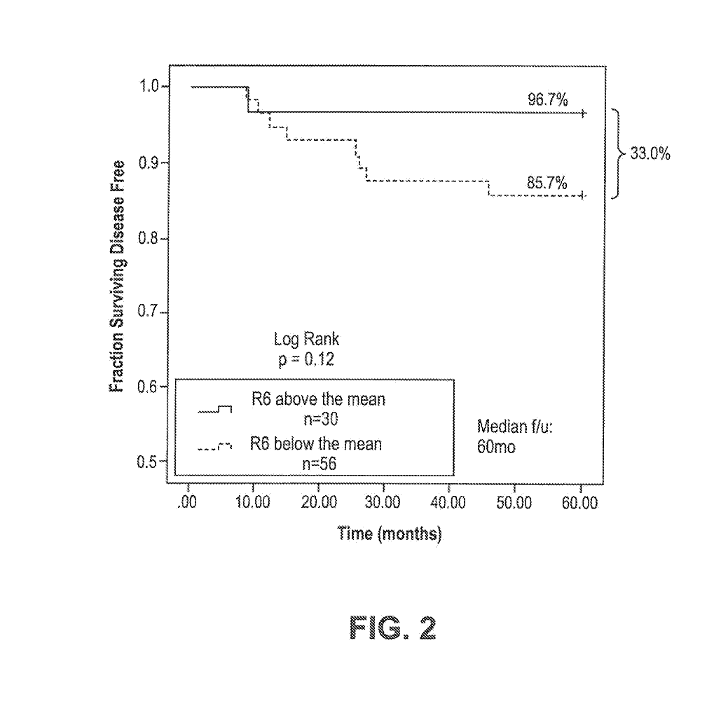

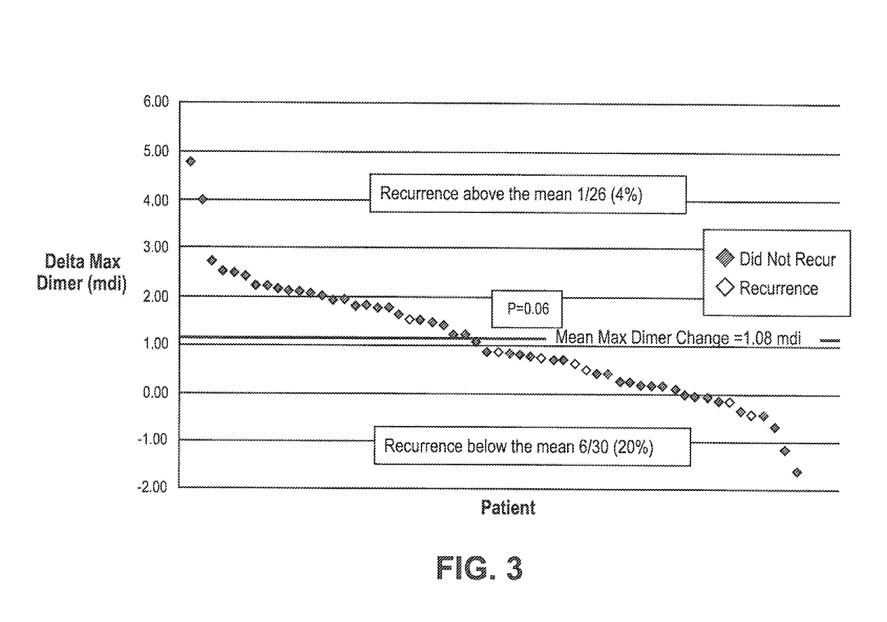

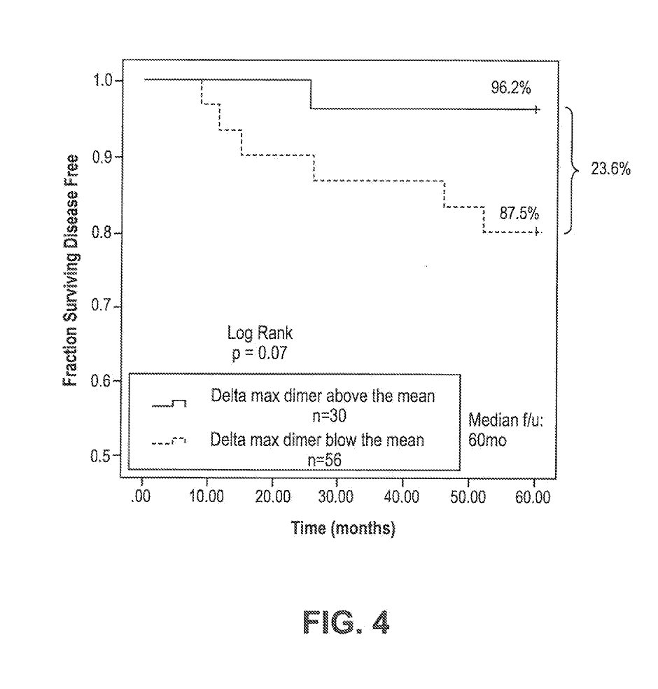

R6 dimer assays were available for 86 patients in the vaccine group. The mean R6 dimer in the vaccine group was 0.63 mdi±0.08. Of the 30 patients with a R6 dimer above the mean, only one recurred, compared to 8 of the 56 below the mean. (p=0.09) (FIG. 1), a relative risk reduction of 33.0%. (FIG. 2) The difference between baseline and maximum mdi was available in 56 HER2 under-expressing, vaccine group patients. Of the 26 patients above the mean difference (1.08 mdi±0.17), one recurred, compared to 6 clinical recurrences in the 30 patients below the mean (p=0.06) (FIG. 3), a relative risk reduction of 23.6%. (FIG. 4) There were no clinical recurrences in patients with HER2 under expression with a mean difference ranked in the top third.

In prospective, completed phase I/II trials of NeuVax (Nelipepimut-S), patients who exhibit robust ex viva immune responses have lower recurrence rates. This finding suggests that E75-specific CTL clonal expansion is a valid biomarker for clinical recurrence in patients treated with E75+GM-CSF.

### EXAMPLE 2

### Use of HLA-A2:Ig Dimer Assay to Predict Clinical Benefit of GP2+GM-CSF Vaccine

A prospective, randomized, multi-center, placebo-controlled, single-blinded, phase II trial was also designed to evaluate the safety and clinical efficacy of an HLA-A2 restricted HER2-derived peptide vaccine, in breast cancer patients.

Clinically disease-free, node positive or high-risk node negative patients with any level of HER2 expression were enrolled after standard of care therapy. Patients received 6 monthly intradermal inoculations (R0-R6) of GP2+GM-CSF during the primary vaccine series followed by four boosters every 6 months. Ex-vivo immune responses were measured by GP2:Ig dimer assay at R0 and R6 and reported as mean dimer index (mdi). Means were compared using student's t-test and portions using Fisher's Exact Test. A LRM with backwards elimination of demographics and dimer assays was used to predict recurrence.

Seventy patients were available for analysis with a median follow-up of 30 months. 65/70 of the patients had an R6 dimer. The mdi significantly increased from R0-R6 (0.81+0.09 mdi v 1.12+0.1 0mdi, p<0.05). No recurrence was observed in patients having an R0 mdi<0.32 (n=23). Recurrence was observed in 6/47 patients having an R0 mdi>0.32 (p=0.16). No recurrence was observed in patients having an R6 mdi>0.61 (n=39), while 4/26 patients with an R6 mdi<0.61 had breast cancer recurrence (p=0.02). There was no difference in demographics between patients who recurred and those who did not. The optimal modeling variables were R0 and R6 dimer:

| Variables in the | Odds Ratio | Sensitivity, | Area Under |
|---|---|---|---|
| Optimal Model | (p-value) | Specificity | the Curve |
| R0 | 1.37 (0.60) | 100%, 34% | 0.62 |
| | | (R0 = 0.32 mdi) | |
| R6 | 2.48 (0.11) | 100%, 65% | 0.80 |
| | | (R6 = 0.61 mdi) | |

As with the E75 analysis in Example 1, lower pre-existing and higher post-vaccination peptide-specific CTL levels correlate with fewer recurrences of cancer in high-risk breast cancer patients, indicating that induction of GP2 immunity correlates with clinical outcome in the adjuvant setting. These correlations have been difficult to prove with vaccines in the metastatic setting.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of monitoring an immune response to a test peptide, the method comprising:
(a) incubating peripheral blood mononuclear cells (PBMCs) obtained from a patient with a fusion protein comprising a mouse immunoglobulin fused to two human MEW class I HLA molecules, wherein the test peptide has been loaded into the peptide binding groove of the two human MEW class I HLA molecules;
(b) quantitating the cytotoxic CD8 T cells specific for the test peptide; and
(c) normalizing the result obtained in step (b) by subtracting a background value obtained by quantitating the cytotoxic CD8 T cells specific for a negative control peptide, wherein the negative control peptide is the E37 peptide having the amino acid sequence RIAWARTEL (SEQ ID NO:4).

2. A method of monitoring an immune response to a test peptide, the method comprising:
(a) incubating peripheral blood mononuclear cells (PBMCs) obtained from a patient with a fusion protein comprising a mouse immunoglobulin fused to two human MEW class I HLA molecules, wherein the test peptide has been loaded into the peptide binding groove of the two human MEW class I HLA molecules;
(b) incubating PBMCs obtained from the patient with the fusion protein, wherein a negative control peptide has been loaded into the peptide binding groove of the two human MHC class I HLA molecules, and wherein the negative control peptide is the E37 peptide having the amino acid sequence RIAWARTEL (SEQ ID NO:4);
(c) quantitating the cytotoxic CD8 T cells specific for the test peptide;
(d) quantitating the cytotoxic CD8 T cells specific for the negative control peptide to obtain a background value;
(e) normalizing the result obtained in step (c) by subtracting the background value obtained in step (d).

3. The method of claim 1, wherein the background value is about 0.3%.

4. The method of claim 1, wherein the test peptide is a class I restricted Her2/neu derived peptide.

5. The method of claim 4, wherein the test peptide is E75, GP2, GP2′, or Her⁵⁷⁷.

6. The method of claim 4, wherein the test peptide is E75.

7. The method of claim 1, wherein the fusion protein comprises a mouse IgG fused to two human MHC class I HLA-A2 molecules.

8. The method of claim 1, wherein quantitating the cytotoxic T cells specific for the test peptide is achieved using immunofluorescent staining or fluorescent activated cell sorting (FACS).

9. The method of claim 1, further comprising a step before (a) of loading the test peptide into the peptide binding groove of the two human MHC class I HLA molecules of the fusion protein.

10. The method of claim 9, wherein the loading step comprises incubating the test peptide with the fusion protein overnight at 37° C.

11. The method of claim 1, wherein quantitating the cytotoxic T cells specific for the test peptide is expressed as a dimer index.

12. The method of claim 1, wherein the method is used to assess the clinical response of a HER2/neu derived peptide vaccine in a breast cancer patient, wherein the breast cancer patient is disease free following standard therapy.

13. The method of claim 12, wherein the HER2/neu derived peptide vaccine comprises E75 and GM-CSF or GP2 and GM-CSF.

14. The method of claim 13, wherein the cytotoxic CD8 T cells specific for the test peptide are quantified at baseline, after a primary vaccination, and at 6 months post primary vaccination.

15. The method of claim 1, wherein a more robust cytotoxic CD8 T cell response to the test peptide at 6 months post primary vaccination indicates that the patient is less likely to experience breast cancer recurrence or more likely to have a longer disease free survival.

16. The method of claim 1, wherein a low cytotoxic CD8 T cell response to the test peptide at baseline indicates that the patient is less likely to experience breast cancer recurrence or more likely to have a longer disease free survival.

17. The method of claim 16, wherein the patient has low to intermediate HER2 expression (IHC 1 or 2+ or FISH<2.2).

18. A kit comprising a fusion protein comprising a mouse immunoglobulin fused to two human MHC class I HLA molecules and a negative control peptide, wherein the negative control peptide is an E37 peptide having the amino acid sequence RIAWARTEL (SEQ ID NO:4).

19. The kit of claim 18, further comprising a positive control peptide.

20. The kit of claim 18, further comprising a test peptide, wherein the test peptide is a class I restricted Her2/neu derived peptide.

21. The kit of claim 20, wherein the test peptide is E75, GP2, GP2′, or Her⁵⁷⁷.

22. The kit of claim 18, wherein the fusion protein comprises a mouse IgG fused to two human MHC class I HLA-A2 molecules.

23. The kit of claim 18, further comprising a labeled antibody that binds to the immunoglobulin of the fusion protein.

24. The kit of claim 18, wherein the E37 peptide is loaded into the peptide binding groove of the two human MHC class I HLA molecules of the fusion protein.

25. The kit of claim 19, wherein the test peptide is loaded into the peptide binding groove of the two human MHC class I HLA molecules of the fusion protein.

26. The kit of claim 18, further comprising a buffer for diluting one or more of the negative control peptide, positive control peptide, or test peptide.


SEQUENCE LISTING (6 sequences)

SEQ ID NO: 1 - protein, 1255 aa, Artificial Sequence [sequence omitted]

SEQ ID NO: 2 - protein, 9 aa, Artificial Sequence
  Description of Artificial Sequence Synthetic peptide
KIFGSLAFL

SEQ ID NO: 3 - protein, 9 aa, Artificial Sequence
  Description of Artificial Sequence Synthetic peptide
IISAVVGIL

SEQ ID NO: 4 - protein, 9 aa, Artificial Sequence
  Description of Artificial Sequence Synthetic peptide
RIAWARTEL

SEQ ID NO: 5 - protein, 9 aa, Artificial Sequence
  Description of Artificial Sequence Synthetic peptide
IVSAVVGIL

SEQ ID NO: 6 - protein, 9 aa, Artificial Sequence
  Description of Artificial Sequence Synthetic peptide
FGPEADQCV